(19) United States
(12) Patent Application Publication
(10) Pub. No.: US 2008/0306580 A1
(43) Pub. Date: Dec. 11, 2008

(54) BLOOD ACESS APPARATUS AND METHOD

(75) Inventors: Mark L. Jenson, Greenfield, MN (US); William J. Drasler, Minnetonka, MN (US); Jason P. Hill, Brooklyn Park, MN (US); Jaydeep Kokate, Plymouth, MN (US); David J. Sogard, Edina, MN (US); Pat Haverkost, Brooklyn Center, MN (US)

Correspondence Address: HOFFMANN & BARON, LLP, 6900 JERICHO TURNPIKE, SYOSSET, NY, 11791, US

(73) Assignee: BOSTON SCIENTIFIC SCIMED, INC., Maple Grove, MN (US)

(21) Appl. No.: 12/025,350

(22) Filed: Feb. 4, 2008

Related U.S. Application Data
(60) Provisional application No. 60/899,601, filed on Feb. 5, 2007.

(51) Int. Cl. A61F 2/82; A61F 2/84; A61B 17/32; B23P 11/00

(52) U.S. Cl. 623/1.11; 623/1.15; 623/1.2; 606/159; 29/428

(57) ABSTRACT

A blood access for dialysis includes a radially distensible support structure having an open lattice structure defining a support wall having interstitial open areas and defining exterior and luminal wall surfaces; a first porous polymeric portion having a plurality of pores, the first porous polymeric portion being securably disposed over at least a portion of the exterior wall portion of the support structure; a second porous polymeric portion having a plurality of pores, the second porous polymeric portion being securably disposed over at least a portion of the luminal portion of the support structure; and a biodegradable and/or bioabsorbable material disposed within the pores of the first polymeric portion and/or pores of the second polymeric portion.

### CROSS-REFERENCE TO RELATED APPLICATIONS

This application claims the benefit of U.S. Provisional Application No. 60/899,601, filed Feb. 5, 2007, the contents of which are incorporated herein by reference.

### FIELD OF THE INVENTION

The invention is related to a blood access device for use in an arteriovenous fistula to provide for blood access for dialysis. More particularly, the present invention is related to a composite material blood access device for dialysis and useful for minimizing arteriovenous fistula maturation time periods, and methods for the same.

### BACKGROUND OF THE INVENTION

Blood access for hemodialysis is commonly achieved by placement of an arteriovenous graft. Typically, an expanded polytetrafluoroethylene (ePTFE) graft is surgically placed in the forearm with one end of the graft anstomosed to an artery and the other end of the graft anstomosed to a vein. Prior to use for blood access, the graft must generally be encapsulated by tissue. Such encapsulation, however, typically takes several weeks, for example about two weeks or more. After tissue encapsulation, the graft may be accessed by direct transcutaneous needle puncture, typically with two dialysis access cannula needles, as often as three times a week.

Such ePTFE blood access grafts, however, generally have poor longevity. Within about six to nine months significant intervention for thrombosis, stenosis and/or infection is often required. Moreover, complete replacement of the graft is often required after about one and a half years. When the graft fails, a new graft is surgically placed at another bodily location, such as an upper arm, a contralateral arm or other location, as needed, to obtain sufficient blood access for continued dialysis treatment.

In contrast to the use of ePTFE blood access grafts, a native fistula may be prepared by surgically anastomosing an artery and a vein, again often in the forearm. Such a native fistula may function as a blood access site for about five years, which is a much longer period as compared to the ePTFE graft. A native fistula, however, requires a long period of maturation, typically several months, before it can be used for blood access.

Patients in the U.S. typically do not get a native fistula early enough prior to their need for dialysis. This is because the native fistula which has a relatively long maturation period. In Europe, native fistulae are more likely to be placed well prior to the dialysis treatment, so that maturation occurs prior to the actual dialysis treatment. Such an advance placement of a fistula, however, requires a very early surgical fistula creation such that the actual need for initiation of dialysis may not be accurately predicted. Further, such advance and early placement often results in that the fistula may be in place much earlier than actually required. Without such advance placement and proper maturation, the use of an alternative access method is often required until the native fistula matures.

Thus, there is a need for a device and method which provides for the superior long-term function of a native fistula, yet provides for earlier access for dialysis similar to the maturation period of an ePTFE graft.

### SUMMARY OF THE INVENTION

Existing synthetic vascular grafts placed as arteriovenous grafts for dialysis applications have many shortcomings. Two major problems are thrombosis, due to lack of proper healing and endothelialization, and intimal hyperplasia, causing luminal narrowing, most commonly at or near the venous anastomosis. Several factors contribute to these problems and are addressed by the devices, systems and methods of the present invention.

Typical vascular grafts are porous, with small interconnecting pores or void spaces which will pass cells and fluids between the inside and outside surfaces so that tissue may grow throughout the graft wall and may cover the inside and outside surfaces of the graft. One goal is to get just the right amount of tissue growth and an endothelial lining on the luminal or interior portion of the graft, so that the antithrombotic activity of endothelium can prevent thrombosis of the vascular graft. However, pore size and structure may be limited by a requirement that the vascular graft not leak blood or plasma in significant amounts in the period after being implanted and prior to maturation. Thus, large pores which facilitate tissue ingrowth are desired, yet small pores which limit leakage are also desired, especially in a structure which may withstand repeated needle puncture for dialysis access.

The present invention overcomes the failings of the prior art by providing a blood access device which can be placed in the vein at the time of native fistula creation. The blood access device provides rapid tissue ingrowth similar to and/or more rapidly than all ePTFE graft. For the months of maturation prior to cannulation of the vein for dialysis, the segment of vein containing the intravascular the blood access device of the present invention may be cannulated. The blood access device of the present invention also provides sufficient visualization of the segment to be cannulated and further provides adequate and/or enhanced sealing of needle puncture sites. The blood access device may be relatively short in length as it need only provide puncture sites for the months of fistula maturation. Since the blood access device is short, problems of thrombosis, infection, hyperplasia, stenosis, and limited endothelialization are advantageously minimized. The blood access device may also provide moderate expansion of the segment of vein, approximately matching the dilation seen in the vein as a native fistula matures, thereby facilitating visual and tactile location of the segment so that the access needles can be placed in the correct location.

To utilize the blood access device of the present invention, a suitable vein may be severed, and the distal end of the vein may be then ligated. The blood access device of the present invention may be inserted into the proximal portion of the vein through its open end and transluminally deployed at a desired location. The open end of the vein is brought to a suitable artery, and anastomosis between the artery and the vein is created. Thus, the procedure is similar to the standard surgical Brescia-Cimino fistula creation, but with the additional key step) of inserting the blood access device into the vasculature. After a short healing period during which tissue grows around the blood access device and into the porous structure of the blood access device, blood access can be achieved such as for dialysis, chemotherapy infusion, or other diagnostic or treatment purpose, by puncture of the vascular segment containing the blood access device. After fistula maturation during which the vein expands and the vein wall thickens, blood access can be achieved by puncture of other portions of the vein as well as the segment containing the blood access device.

The blood access device has a porous structure which facilitates rapid and complete tissue ingrowth. Since the blood access device is to be placed within the vasculature, leakage of blood through the porous structure is not a problem at implant. After tissue ingrowth into the porous structure, the tissue prevents leakage of blood through the porous structure so that even when the vein is punctured for blood access, the needle tract will seal with a short period of compression.

In one aspect of the present invention, the blood access device includes a porous polymer such as Styrene-Isobutylene-Styrene (SIBS) polymer or SIBS-coated ePTFE which facilitates rapid tissue ingrowth (typical pore size 40-150 micrometer preferred) yet provides sufficient structure to hold the healed device together and provide for sealing of the needle puncture sites. A preferred structure for the blood access device includes an expansile element or support element which provides good apposition of the device to the vein wall, moderate dilation of the blood access device and vein segment containing the blood access device, tactile feedback facilitating location and puncture of the veins segment blood access device and resilience against any external crushing force. The expansile element may be a metallic structure such as wire windings or braid, slotted tube, or other formed or deposited metal element which provides expansile force and has open structure to facilitate tissue ingrowth through the structure. The porous polymer and expansile element are bonded by adhesion and/or mechanical interlock such as encapsulation or surrounding of at least portions of the expansile element by polymer, which can be the same polymer as the porous polymer structure, or a separate bonding polymer. Optionally, one or more portions of the blood access device can include an agent, such as a therapeutic agent, for example, the polymer used in the porous polymer structure may have an agent which reduces cellular proliferation, such as paclitaxel, incorporated to reduce hyperplasia and stenosis development. Other agents known in the art can also be incorporated as described below.

The short length, porous structure, and intravascular placement of the blood access device of the present invention provide superior utility without any added drug or agent. To further enhance the healing and function of the blood access device or the adjacent vein segments, the blood access device may also be configured to provide a drug elution capability so that agents such as growth factors, thrombosis inhibitors, platelet inhibitors, inflammatory inhibitors, cellular proliferation or migration modifying agents, or other agents may be included. Surface adsorption of these agents, binding agents, proteins or ligands, cells, or cellular precursors may also be accomplished due to the unique characteristics of the present invention. Agents may be included in selected portions of the blood access device or the entire blood access device, and the blood access device may be configured to retain the agent(s), or release them over a short or long duration depending on the particular effects desired. For example, anticoagulant or antiplatelet agents may be applied selectively to the luminal surface of the entire blood access device, agents that stimulate endothelial proliferation and migration may then be applied selectively to the subluminal portion away from the ends of the blood access device, and cellular proliferation inhibitor agents may be applied selectively to one or both ends of the blood access device, or a combination can be applied, with similar or varying duration of activity or elution rates. Regardless of the choice of any agents, the porous structure allows tissue ingrowth through the wall of the blood access device along the entire length of the blood access device. The porous structure allows tissue ingrowth through every portion of the wall, or selected intermittent regions of the blood access device may allow tissue ingrowth as long as the intermittent regions are present along the entire length of the blood access device and are not spaced too far apart.

In another aspect of the present invention, a blood access device includes a first layer of porous SIBS, which may be constructed or formed by electrostatic spinning. A wire braid may be applied to the first layer of SIBS and may slightly compress the layer of SIBS under the wire(s). A second layer of porous SIBS may be constructed or formed, capturing or encapsulating the wire braid to provide a strong and unitary structure. One or both layers of SIBS may include an agent, or additional polymer with agent may be applied, or agent may be applied to the surface(s).

The present invention also includes methods of fabricating a blood access device. The present invention also includes methods of treating a patient.

The present invention may also include the use of other polymers, other strengthening materials, or other biologically active materials.

The present invention may also include the use of biologically active material to reduce infections, reduce inflammation, reduce thrombosis, or encourage healing, or encourage endothelialization of the blood access device.

Further, the blood access device of the present invention may include additional layers that may be used for controlling leakage or enhancing the useability or performance of the blood access device.

The blood access device of the present invention may, also be placed elsewhere in the vasculature. Multiple blood access devices may be used. Two blood access devices may be used in contralateral veins, one for withdrawing blood and the other for infusing blood.

These and other aspects, objectives, features ad advantages of this invention will become apparent from the following detailed description of illustrative embodiments thereof, which is to be read in connection with the accompanying drawings in which like reference characters refer to the same parts or elements throughout the different views. The drawings are not necessarily to scale, emphasis instead being placed upon illustrating the principles of the invention.

### BRIEF DESCRIPTION OF THE DRAWINGS

FIG. 1 is a perspective view of a blood access device of the present invention.

FIG. 2 is a cross-sectional view of the blood-access device of FIG. 1 taken along the 2-2 axis.

FIG. 3 is a cross-sectional view of the blood-access device of FIG. 1 taken along the 3-3 axis.

FIG. 4 is a perspective view of the blood-access device of FIG. 1 depicting a porous polymeric portion of the device.

FIG. 5 is an exploded view of a portion of the porous polymer portion of FIG. 4.

FIG. 6 is another exploded view of a portion of the porous polymer portion of FIG. 4.

FIG. 7 is another exploded view of a portion of the porous polymer portion of FIG. 4.

FIG. 7 is another exploded view of a portion of the porous polymer portion of FIG. 4.

FIG. 8 is another exploded view of a portion of the porous polymer portion of FIG. 4.

FIGS. 9-11 depict alternate embodiments of porous polymeric structures of the present invention.

FIGS. 12-13 depict alternate embodiments of the blood access device of FIG. 1 taken along the 3-3 axis.

FIGS. 14-23 depict alternate embodiments of a radially expandable support of the blood access device of the present invention.

FIGS. 24-25 depict use or methods of implanting the blood access device of the present invention.

### DETAILED DESCRIPTION OF PREFERRED EMBODIMENTS

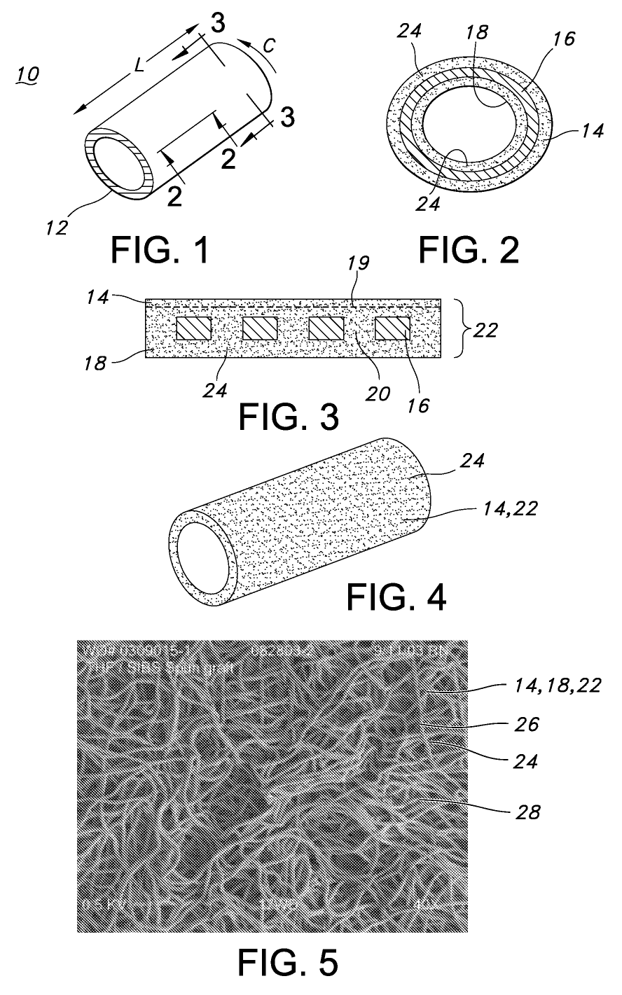

FIG. 1 is a perspective view of a blood access device 10 of the present invention. The blood access device 10 is a single lumen device defined by a cylindrical wall 12. FIG. 2 is a cross-sectional view of the of the blood access device 10 of FIG. 1 taken along the 2-2 axis. The blood access device 10 includes an external polymeric portion 14, an expandable support structure 16, and an internal or luminal polymeric portion 18. The external polymeric portion 14 and the luminal polymeric portion 18 may be the same or different. Further, the external polymeric portion 14 and the luminal polymeric portion 18 may be a unitary structure formed by the same or similar materials at or about the same time by a similar formation technique. For example, the luminal polymeric portion 18 may be formed or disposed over a mandrel (not shown), which is typically a cylindrical mandrel. The expandable support structure 16 may then be formed or disposed over the luminal polymeric portion 18. The external polymeric portion 14 may then be formed or disposed over the expandable support structure 16 and/or the luminal polymeric portion 18. Alternatively, the external polymeric portion 14 and the luminal polymeric portion 18 may be formed or disposed as separate layers and securably joined to one and the other by chemical means, such as through the use of adhesives and the like, through mechanical means, such as suturing the portions together and/or optionally suturing the layers to the support structure and the like, through pressure means, and/or through thermal means. Useful adhesives and techniques for using adhesives for securing components of implantable vascular devices may be found in U.S. Patent Application Publications Nos. 2003/10139806 A1 to Haverkost et al., 2003/0017775 A1 Sowinski et al. and 2004/0182511 A1 to Rakos et al., the contents of which are incorporated herein by reference.

FIG. 3 is a cross-sectional view of the blood access device 10 of FIG. 1 taken along the 3-3 axis. Desirably, the luminal polymeric portion 18 encapsulates the expandable support structure 16, including the interstitial spaces, openings or areas 20. The external polymeric portion 14 may then be disposed over the luminal polymeric portion 18 or portions of the luminal polymeric portion 18. Further, as described below, the luminal polymeric portion 18 and the external polymeric portion 14 may be formed as or into a unitary polymeric portion 22.

Desirably, the external polymeric portion 14, the luminal polymeric portion 18, and/or the unitary polymeric portion 22 are a porous portion or structure. Useful porosities include, but are not limited to, a pore size of greater than about 10 microns (i.e., micrometers or μm), for example about 10 microns to about 150 microns. Useful pore sizes also include pore sizes from about 40 microns to about 150 microns and less than about 50 microns. A pore size from about 1 micron to about 10 microns or larger may also be used. Such pores are depicted as element 24 in FIGS. 2-4. Desirably, the external polymeric portion 14, as depicted in FIG. 4, includes the porous portion having the pores 24.

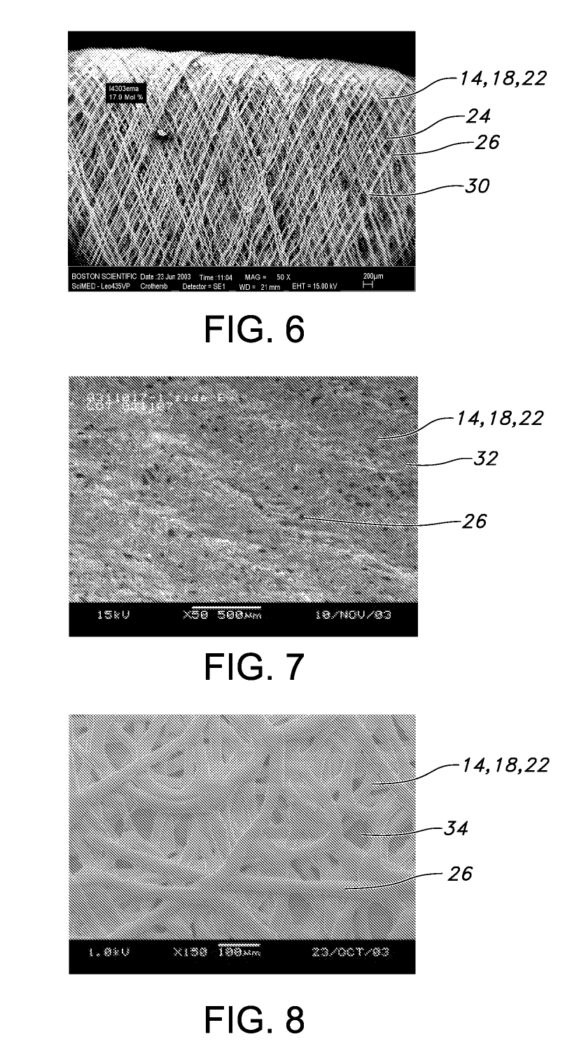

As depicted in FIG. 5, in one aspect of the present invention, the external polymeric portion 14, the luminal polymeric portion 18, and/or the unitary polymeric portion 22 may be a filament spun or multifilament spun portion 24. One useful, but non-limiting, technique for forming the filament spun or multifilament spun portion 24 includes electrostatic spinning of a filament over a substrate, such as a mandrel, to create an electrostatic spun (ELS) spun portion 24. The filaments 26 may have any useful filament diameter, including, but not limited, to filament diameters of about 1 micron to about 50 microns, including from about 5 microns to about 15 microns. The filaments 26 may be spun, or otherwise disposed, in a random pattern 28, including a somewhat random pattern, a substantially random pattern, an approximately random pattern, and the like, as depicted in FIG. 5. The filaments 26 need not, however, be spun in a random pattern 28. For example, as depicted in FIG. 6, the filaments 26 may be spun without electrostatics in an organized pattern 30, including a somewhat organized pattern, a substantially organized pattern, awl approximately organized pattern, and the like. The filaments 26 may be spun from a spinneret or spinnerets which rotate about a substrate, for example a mandrel, including a cylindrical mandrel, may be spun from rotatable or moveable spinneret or spinnerets which rotate or move about a substrate, for example a cylindrical mandrel, or combinations thereof. Moreover, single filaments or multifilaments, which may be the same or different may be spun to form the blood access device of the present invention. Further details of such electrostatic spun portions or grafts and techniques for forming the same may be found in U.S. Pat. No. 4,738,740 to Pinchuk et al., the contents of which are incorporated herein by reference. Moreover, the filaments 26 may be suitably disposed in two-dimensional and/or three-dimensional random or organized patterns from applicators onto a suitable substrate. Details of a system having such applicators and biocompatible substrates formed therefrom may be found in U.S. Pat. No. 7,083,697 to Dao et al., the contents of which are incorporated herein by reference.

Natural polymers, synthetic polymers, or combinations thereof may be used. It is possible, for example, to use a mixture of a non-fiber forming polymer and a fiber forming polymer, wherein the non-fiber forming polymer is present in a small enough percentage of the total mixture to impart desired properties, while still allowing formation of a fiber for application onto the moveable table. The polymers may be biodegradable, biostable, or combinations thereof. Biodegradable synthetic polymers include, but are not limited to, poly α-hydroxy acids such as poly L-lactic acid (PLA), polyglycolic acid (PGA) and copolymers thereof (i.e., poly D,L-lactic co-glycolic acid (PLGA)), and hyaluronic acid. Non-limiting examples of some useful biodegradable natural polymers include polysaccharides such as alginate, cellulose, dextran, polyhyaluronic acid, chitin, poly(3-hydroxyalkanoate), poly(3-hydroxyoctanoate) and poly(3-hydroxyfatty acid), chemical derivatives therefrom, and combinations thereof. As used herein, “biodegradable” materials are those which are broken down and/or absorbed by the body. Examples include materials containing bonds that may be cleaved under physiological conditions, including enzymatic or hydrolytic scission of the chemical bonds, or may be absorbed by the body. Non-limiting useful synthetic polymers include olefin polymers including polyethylenes, polypropylenes, polyvinyl chlorides, polytetrafluoroethylene, expanded polytetrafluoroethylene, polyvinyl acetates, polystyrenes, poly(ethylene terephthalate), polyurethanes, polyether polyurethanes, polyester polyurethanes, polycarbonate polyurethanes, polyureas, silicone rubbers, polyamides, polycarbonates, polyaldehydes, natural rubbers, polyether-ester copolymers, styrene-butadiene copolymers, poly(vinyl alcohols), polyamides, polyester amides, poly(amino acids), polyanhydrides, polyacrylates, polyalkylenes, polyalkylene glycols, polyalkylene oxides, polyalkylene terephthalates, polyortho esters, polyvinyl ethers, polyvinyl esters, polyvinyl halides, polyvinylpyrrolidone, polyesters, polylactides, polyglyxolides, polysiloxanes, polycaprolactones, polyhydroxybutrates, styrene isobutyl styrenes, styrene isobutyl styrene block polymers, copolymers, block polymers and combinations thereof.

Desirably, the filaments 26, the external polymeric portion 14, the luminal polymeric portion 18, and/or the unitary polymeric portion 22 include elastomeric materials or polymers. Useful non-limiting elastomeric materials include styrene isobutylene styrenes, natural rubbers, silicones, polyurethanes, and the like. Desirably, the filaments 26, the external polymeric portion 14, the luminal polymeric portion 18, and/or the unitary polymeric portion 22 include elastomeric styrene isobutyl styrene polymers or copolymers.

As described above, the filaments 26, the external polymeric portion 14, the luminal polymeric portion 18, and/or the unitary polymeric portion 22 may be disposed or formed at various useful porosities. FIGS. 7 and 8 depict substrate portions having different porosities. For example, the filaments 26, the external polymeric portion 14, the luminal polymeric portion 18, and/or the unitary polymeric portion 22 may have smaller pore size 32 as depicted in FIG. 7 or a larger pore size 34 as depicted in FIG. 8.

The present invention, however, is not limited to the external polymeric portion 14, the luminal polymeric portion 18, and/or the unitary polymeric portion 22 formed by electrostatic depositing techniques, for example electrostatic spinning, and other techniques for forming or providing porous polymeric portions 14, 18, 22 may suitably be used. For example, the above-described materials may be extruded, sprayed, dipped, coated, cast, and the like to form porous substrates, including cylindrical substrates. Porosity may be introduced into the formed substrates by the including of a removable non-polymeric material. For example, the substrate-forming material may be co-extruded, co-sprayed, co-dipped, co-coated, co-cast, and the like with a solvent material. After forming the substrate, the solvent evaporates and thereby forms the porous polymeric and/or porous elastomeric substrate. The present invention, however, is not limited to the use of evaporative solvents for forming porous polymeric and/or porous elastomeric substrates, and other techniques for forming may suitably be used. For example, the substrate-forming material may be co-extruded, co-sprayed, co-dipped, co-coated, co-cast, and the like with leachable material, such as a salt, which may suitable be removed, for example by washing, to thereby form the porous polymeric and/or porous elastomeric substrate.

Furthermore, the porous polymeric and/or porous elastomeric substrate may also suitably be formed by textile techniques. As used herein, the term “textile” refers to a material, such as a filament or yarn, that has been knitted, woven, braided and the like into a structure, including a hollow, tubular structure. As used herein, the term “non-textile” and its variants refer to a material formed by casting, molding, spinning or extruding techniques to the exclusion of typical textile forming techniques, such as braiding, weaving, knitting and the like. Any of the above-described substrate-forming materials may suitably be used to form a textile substrate which may function as the porous polymeric portions 14, 18, 22.

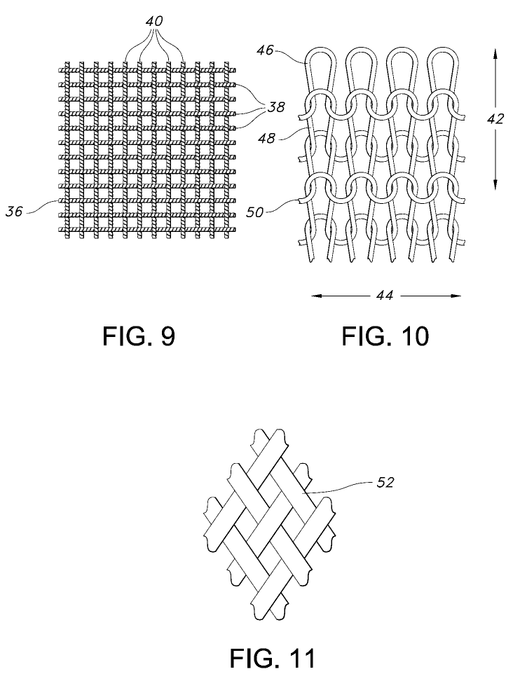

The textile portion of the present invention can have virtually any textile construction, including weaves, knits, braids, filament windings and the like. As depicted in FIG. 9, a useful textile portion includes a woven textile. Useful weave patterns include simple weaves, basket weaves, twill weaves, satin weaves, velour weaves and the like. The weave pattern 36 for the woven portion includes warp filaments 38 running along the longitudinal length (as indicated by vector L in FIG. 1) of the woven product and fill filaments 40 running around the circumference (as indicated by vector C in FIG. 11) of the product the warp, the fill filaments being at approximately 90 degrees to one another with fabric flowing from the machine in the warp direction.

The textile portion may also be a knitted textile portion. Knitting involves the interlooping or stitching of filaments into vertical columns (wales) and horizontal rows (courses) of loops to form the knitted fabric structure. Warp knitting is particularly useful with the knitted textile portions of the present invention. In warp knitting, the loops are formed along the textile length, i.e., in the wale or warp direction of the textile. As depicted in FIG. 10, for a tubular textile, such as blood access device 10, stitches in the axial or longitudinal direction (L) of the tubular textile are called wales (indicated by vector 42 in FIG. 10) and stitches in the radial or circumferential direction (C) of the tubular textile are called courses (indicated by vector 44 in FIG. 10). Filaments 46 and 48 interloop in the warp direction to form a warp-knitted pattern 50. Useful warp-knitted patterns include high-stretch patterns and warp-knitted patterns. Useful, but not limiting, examples of high-stretch patterns include those with multiple patterns of diagonally shifting filaments including modified atlas knits as described in U.S. Pat. No. 6,540,773, the contents of which are in incorporated herein by reference, and warp knitted patterns including multiple needle underlap and one needle overlap, such as those patterns described in U.S. Pat. No. 6,554,855, the contents of which are incorporated herein by reference. Useful, but not limiting, examples of warp-knitted patterns, such as locknit (also referred to as tricot or jersey knits), reverse locknit, sharkskin, queenscord and velour knit patterns.

Braiding may also be used as shown, for example, in FIG. 1. Useful braids include, but are not limited to, a diamond braid having a 1/1 intersection repeat (i.e., braid 52 as depicted in FIG. 11), a regular braid having a 2/2 intersection repeat (not shown), or a Hercules braid having a 3/3 intersection repeat (not shown). U.S. Pat. No. 5,653,746, the content of which is incorporated herein by reference, further describes such braids. Moreover, a triaxial braid may also be used. A triaxial braid has at least one filament that typically runs in the longitudinal direction or axial direction of the textile portion to limit filament movement. The axial or longitudinal filament is not interlaced or interwound with the other braid filaments, but is trapped between the different sets of filaments in the braided structure. Moreover, an interlocking three-dimensional braided structure or a multi-layered braided structure is also useful. A multi-layered braided structure is defined as a structure formed by braiding wherein the structure has a plurality of distinct and discrete layers.

Braiding machines, including circular braiding machines that form a braided textile over a mandrel, are useful with the practice of the present invention. An example of such a braiding machine is described in U.S. Pat. No. 6,652,571, the content of which is incorporated herein by reference. A braiding machine capable of forming the interlocked three-dimensional braid used to form the textile tube of the present invention is described in International Patent Publication No. WO 91/10766, which is incorporated herein by reference.

These textile structures may also be composite structures. For example, composite textile structures may include more than one type of textile material, and/or include a varied textile filament diameter or profile, include varied filament spacing to, for example, achieve an appropriate balance among strength and kink-resistance and the prevention of plasma weeping. Further, the external polymeric portion 14, the luminal polymeric portion 18, and/or the unitary polymeric portion 22 may also be composites.

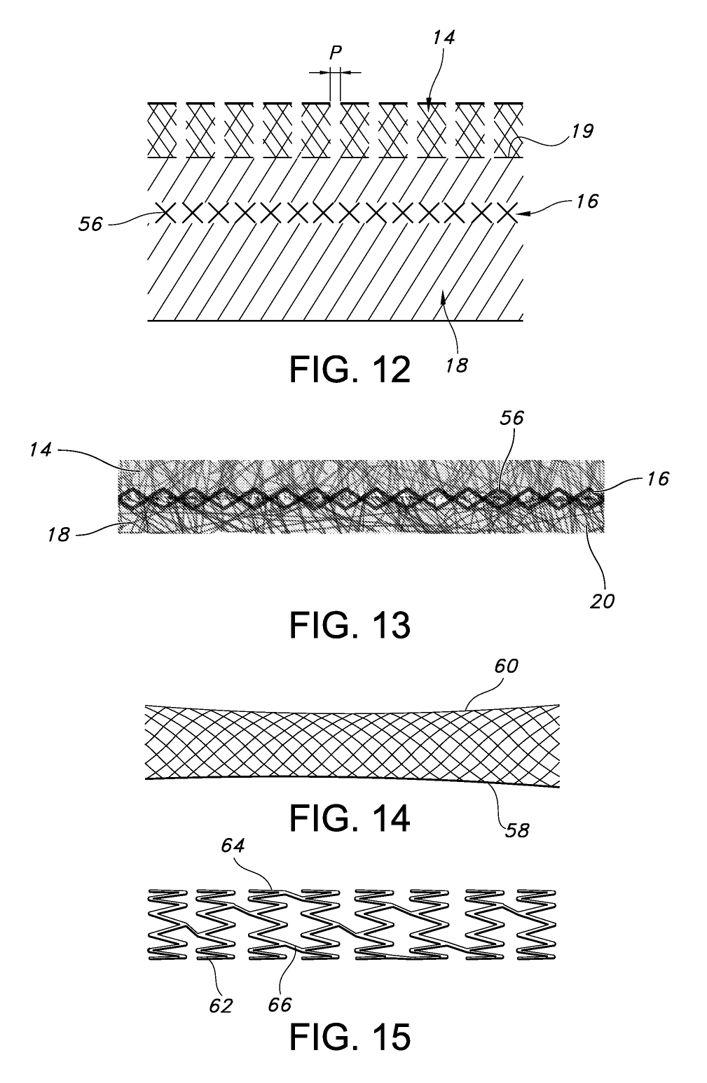

FIGS. 12 and 13 are additional depictions of a portion of the blood access device 10 of the present invention. As depicted in FIG. 12, the expandable support structure 16 may include support elements, filaments or wires 56. Desirably, the expandable support structure 16 is a radially distensible structure, more desirably a self-expanding radially distensible structure. The external polymeric portion 14 may include, as described below, a material, such as a biodegradable material, within the pores 24. As depicted in FIG. 12, the resulting pore size “P” is effectively reduced after having a biodegradable material disposed in the pores 24. The pore size “P” may be in the order of about one micron. Such a pore size is non-limiting. FIG. 13 depicts filament spun porous polymeric portions 14, 18 of the present invention. The porous polymeric portions 14, 18 may be the same or different.

Desirably, the expandable support structure 16 and/or the support elements, filaments or wires 56 are made from any suitable implantable material, including without limitation, nitinol, stainless steel, cobalt-based alloy such as Elgiloy®, platinum, gold, titanium, tantalum, niobium, polymeric materials and combinations thereof. Useful and nonlimiting examples of polymeric stent materials include poly(L-lactide) (PLLA), poly(D,L-lactide) (PLA), polyglycolide) (PGA), poly(L-lactide-co-D,L-lactide) (PLLA/PLA), poly(L-lactide-co-glycolide) (PLLA/PGA), poly(D,L-lactide-co-glycolide) (PLA/PGA), poly(glycolide-co-trimethylene carbonate) (PGA/PTMC), polydioxanone (PDS), Polycaprolactone (PCL), polyhydroxybutyrate (PHBT), poly(phosphazene) poly(D,L-lactide-co-caprolactone) PLA/PCL), poly(glycolide-co-caprolactone) (PGA/PCL), poly(phosphate ester) and the like. Desirably, the expandable support structure 16 and/or the support elements, filaments or wires 56 comprise nitinol.

Various support structures 16 and support structure constructions may be employed in the invention. Useful support structures 16 include, without limitation, self-expanding support structures and balloon expandable support structures. Desirably, the support structures 16 include, without limitation, self-expanding support structures. The support structures 16 may be capable of radially contracting or expanding, as well, and in this sense can be best described as radially or circumferentially distensible or deformable. Self-expanding support structures 16 include those that have a spring-like action which causes the support structures 16 to radially expand, or support structures 16 which expand due to the memory properties of the stent material for a particular configuration at a certain temperature. Nitinol is one material which has the ability to perform well while both in spring-like mode, as well as in a memory mode based on temperature. Other materials are of course contemplated, such as stainless steel, platinum, gold, titanium and other biocompatible metals, as well as polymeric materials. The configuration of the support structures 16 may also be chosen from a host of geometries. For example, wire support structures can be fastened into a continuous helical pattern, with or without a wave-like or zig-zag in the wire, to form a radially deformable support structures. Individual rings or circular members can be linked together such as by struts, sutures, welding or interlacing or locking of the rings to form a tubular support structures. Tubular support structures useful in the present invention also include those formed by etching or cutting a pattern from a tube. Such support structures are often referred to as slotted support structures. Furthermore, support structures may be formed by etching a pattern into a material or mold and depositing stent material in the pattern, such as by chemical vapor deposition or the like. Examples of various stent configurations are shown in U.S. Pat. Nos. 4,503,569 to Dotter; 4,733,665 to Palmaz; 4,856,561 to Hillstead; 4,580,568 to Gianturco; 4,732,152 to Waallsten, 4,886,062 to Wiktor, 5,876,448 to Thompson, 5,662,713, to Andersen et al., and 6,264,689 to Colgan et al., all of whose contents are incorporated herein by reference.

As shown in FIG. 14, a filament support structure 58 is a hollow tubular structure formed from filament strand 60 or multiple wire strands 60. Filament support structure 58 may be formed by, for example, braiding or spinning wire filament(s) 60 over a mandrel (not shown). Filament support structure 58 is capable of being radially compressed and longitudinally extended for implantation into a bodily lumen. The degree of elongation depends upon the structure and materials of the filament support structure 58 and can be quite varied, for example, about 5% to about 200% of the length of Filament support structure 58. The diameter of Filament support structure 58 may also become several times smaller as it elongates.

A zig-zag filament support structure 62 is also useful as the support structure 16. Filament strand 64 is being arranged in what can be described as a multiple of “Z” or “zig-zag” patterns to form a hollow tubular support structure. The different zig-zag patterns may optionally be connected by connecting member 66. Further, zig-zag filament support structure 62 is not limited to a series of concentric loops as depicted in FIG. 15, but may be suitably formed by helically winding of the “zig-zag” pattern over a mandrel (not shown).

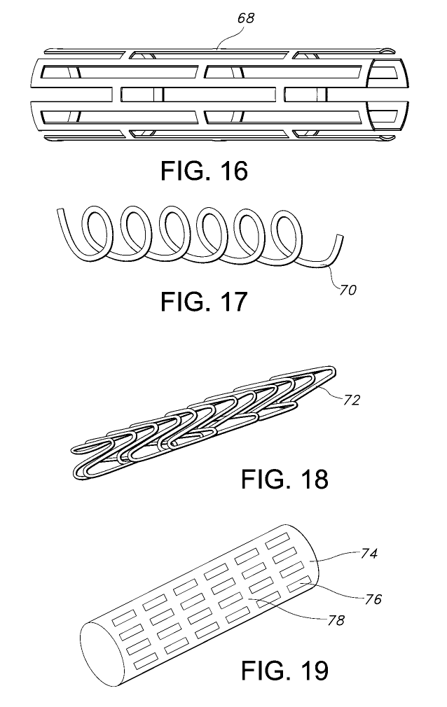

A slotted support structure 68 is also useful as part of the blood access device 10. As depicted in FIG. 16, slotted support structure 68 may be suitably configured for implantation into a bodily lumen (not shown). Upon locating the slotted support structure 68 at the desired bodily site, slotted support structure 68 is radially expanded and longitudinally contracted for securement at the desired site.

Other useful support structures capable of radial expansion are depicted in FIGS. 17 and 18. As depicted in FIG. 17, support structure 70 may be a helical coil which is capable of achieving a radially expanded state (not shown). Support structure 72, as depicted in FIG. 18, has an elongate pre-helically coiled configuration as shown by the waves of non-overlapping undulating windings. These helically coiled or pre-helically structures, commonly referred to as nested structures, are also useful with the practice of the present invention.

The above-described support structures 58, 62, 70, 72 may be referred to as filament-type structures as they as typically formed from elongate filaments. The slotted structure 68 is generally not formed from a plurality of individual elongate elements, but is typically formed by machining, molding, depositing, and the like.

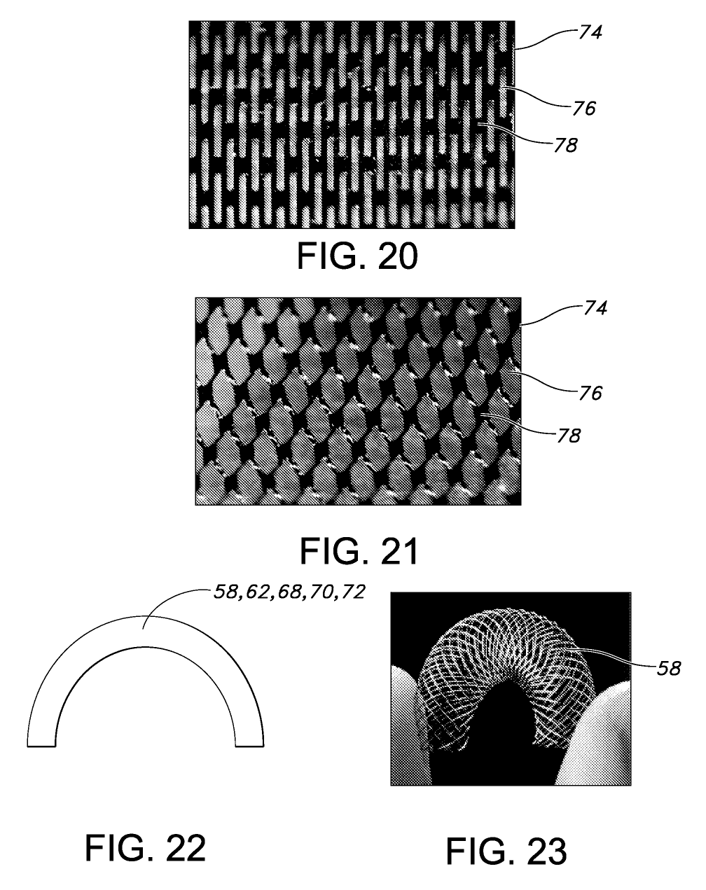

FIGS. 19-21 depict another embodiment of a support structure 74 according to the present invention. Support structure 74 may be referred to as a mesh or fenestrated support structure. The support structure 74 may be made from a thin film or foil, including planar, flat, curved and cylindrical films or foils. As depicted in FIGS. 19-21, the support structure 74 includes a plurality of mesh openings 76. The mesh openings 76 represent the interstitial openings among the support material 78. FIG. 20 is an exploded top planar view of a portion of the support structure 74 of FIG. 19. The mesh openings 76 are depicted as a series of narrow slots. The support structure 74 of FIG. 20 is in an unexpanded or quiescent state. The support structure 74 of FIG. 21 is in a radically expanded state. In the expanded state, the mesh openings are enlarged. Although, the support structure 74 may be made from any of the above-described materials, particularly from any of the above-described metal or metallic materials, desirably the support structure 74 is made from, or includes nitinol. Further, the support structure 74 may be a self-expanding structure. The support structure 74 may be formed by vapor deposition, photolithography, chemical etching, electrochemical etching, mechanical processing or cutting, laser cutting, and the like. Desirably, the foil, film or substrate forming the support structure 74 is a thin foil, film or substrate. Useful methods for forming implantable tubular devices by vapor deposition, chemical etching and/or electrochemical etching may be found in U.S. Pat. Nos. 5,772,860 to Møller et al. and 6,938,668 to Whicher et al., the contents of which are incorporated herein by reference. If the support structure 74 is formed from a flat or planar foil or film, the fenestrated foil or film may be rolled into a tubular structure to form a tubular support structure. The portions of foil or film may be secured to one and the other to securably form a tubular structure. Alternatively, or in addition to, the above-described encapsulation by the porous polymer may also serve to hold the support structure in a tubular or substantially tubular shape.

From the depictions, it may appear that some of the support structures of the present invention may be considered similar to devices commonly referred to as stents. The support structures of the present invention, however, differ significantly from known stents. Stents are often used in bodily lumens to open the lumen. Support structures of the present invention do not need to have such a large radial or hoop strength as the support structures of the present invention do not necessarily function to hold open a damaged vessel. Rather, the support structures of the present invention only need sufficient radial or hoop strength to snug the blood access device 10 of the present invention against a vessel wall, such as a vein. As a result of the support structures of the present invention are more flexible, pliable and bendable than comparable stent devices. The support structures of the present invention, especially support structure 74, have may a thickness from about 0.0005 inches (0.01 mm) to about 0.008 inches (0.2 mm). Desirably, the thicknesses are from about 0.001 inches (0.03 mm) to about 0.004 inches (0.1 mm), more desirably from about 0.002 inches (0.05 mm) to about 0.003 inches (0.08 mm). Stent wires are generally thicker a diameter from a minimum of about 0.004 inches (0.1 mm) to about 0.008 inches (0.2 mm), or thicker.

Desirably, the overall profile of the blood access device 10, including the support structure and the polymeric portion or layers, is also very thin. The overall profile or wall thickness of the blood access device 10 may from about 50 microns to about 1.5 mm, desirably from about 0.1 mm to about 0.5 mm, in particular from about 0.2 mm to about 0.3 mm. Such profiles are, however, nonlimiting and other profiles, including thinner profiles, may suitably be used. Moreover, the profile of the blood access device 10 may vary. For example, the profile of the blood access device 10 may be lower at the interstitial areas of the support structure or the profile may be larger at selected portions, such as the terminal ends, to aid in securement of the device within a bodily lumen.

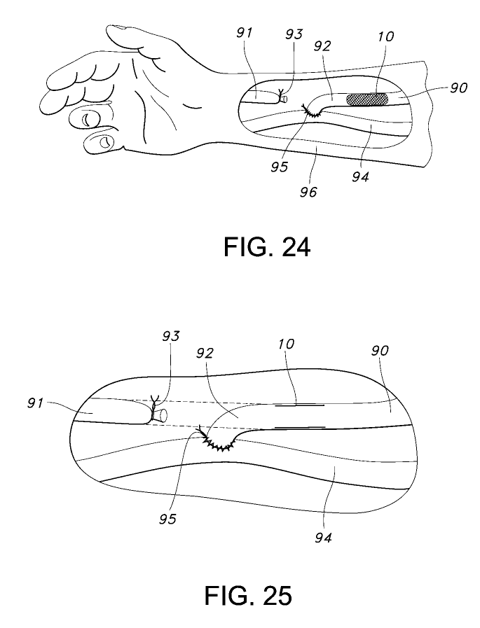

FIGS. 24-25 depict placement and/or use of the blood access device 10 within a bodily lumen of a patient. A bodily lumen, such as a vein 90 is cut or severed into two portions 91, 92. The open end of one vein portion 91 is closed or ligated to form a closed end 93 of the vein portion 91. The blood access device 10 is inserted through the open end of the second vein portion 92. The open end of the second vein portion 92 is anastomosed to an artery 94 at anastomosis 95. Although the blood access device 10 is depicted in FIG. 24 as being placed with the forearm of a patient, the present invention is not so limited, and the blood access device 10 may suitably be disposed at other bodily locations, including those location disclosed in U.S. Provisional Patent Application No. 60/899,602, entitled “Expandable Dialysis Apparatus and Method”, attorney docket number 760-283P, filed Feb. 5, 2007, the contents of which are incorporated herein by reference.

The blood access device 10 depicted in FIGS. 25-26 is relatively short. A nonlimiting short length is from about one inch (about 3 cm) to about three inches (about 7 cm). The use of such a nonlimiting short length allows a practitioner to access the vein through the blood access device 10 within only about one week after implantation. Thus, a patient in need of dialysis may receive dialysis through the implanted blood access device 10 even before the arteriovenous fistula matures. Such a one-week time period is considerably shorter than time periods associated with the techniques of the prior art.

The present invention, however, is not limited to the use of such short lengths of the blood access device 10. For example, the blood access device 10 may be considerably longer so that it is implanted over a much longer portion of the vein or bodily lumen. For example, the blood access device 10 with a length of about 4 inches (about 10 cm) to about 8 inches (about 20 cm)₃ including a length of about 6 inches (about 15 cm) may suitably be used or implanted over a much larger portion of the vein. The vein may then be accessed only through the blood access device 10. Such a longer length of the blood access device 10 minimizes the weakening of the vein over time due to puncturing over time by dialysis needles. Further, with the longer length of the blood access device 10 it is possible to deliver drugs or therapeutic agents over the entire access region.

Another important aspect of the support structures of the present invention is that the structures have sufficient material strength and mesh opening or interstitial opening size such that a needle or a cannula does not cut through the support structure itself. Generally for dialysis treatment, needles or cannulas of about 15 to 16 gauge are used. Accordingly the mesh opening or the interstitial spacing of the support structures in the expanded state should be about 1.5 mm to about 2 mm, or larger. A particularly useful blood withdrawing device with a reduced profile and reduced trauma is disclosed in U.S. Provisional Patent Application No. 60/899,602, entitled “Expandable Dialysis Apparatus and Method”, attorney docket number 760-283P, filed Feb. 5, 2007, the contents of which are incorporated herein by reference.

The support structures 58, 62, 68, 70, 72, 74 of the present invention may have varying geometry. The terminal portions of the support structures 58, 62, 68, 70, 72, 74 may have a higher hoop or radial strength or greater dimension than the remaining portions of the structures. The terminal portions could be thicker and/or have a larger diameter than the other portions of the structure. Further, different types of the above-described structures could be combined to also vary the profile and characteristics of the resulting support structure. Moreover, the support structure may include a plurality of support structures, either proximally or juxtaposingly disposed or spaced apart from one and the other.

Due to their construction, including their thinness, the support structures 58, 62, 68, 70, 72, 74 of the present invention are very bendable and/or flexible, as depicted in FIGS. 22-23.

As described above, the pores 24 of the porous polymeric portions 14, 18, 22 may be filled with a material. For example, the pores 24, especially the pores 24 of the external polymeric portion 14, may be filled with a biodegradable or bioabsorbable material. The biodegradable or bioabsorbable material may include extracellular matrix (ECM) material derived from porcine urinary bladder (UBM), from the urinary bladder matrix (UBM), small intestinal submucosa (SIS), and the like. Other useful materials include, proteins, such as casein, gelatin, gluten, zein, modified zein, serum albumin and collagen, polysaccharides, such as alginate, chitin, celluloses, dextrans, pullulan, and polyhyaluronic acid; poly(3-hydroxyalkanoate)s, poly(β-hydroxybutyrate), poly(3-hydroxyoctanoate) and poly(3-hydroxyfatty acids). Such material also promote tissue ingrowth which further serves to reduce maturation time of the blood access device 10 and also aids in the resealbility characteristic of the blood access device 10 of the present invention. Useful and nonlimiting examples of additional bioabsorbable or biodegradable polymeric materials include poly(L-lactide) (PLLA), poly(D,L-lactide) (PLA), poly(glycolide) (PGA)₅ poly(L-lactide-co-D,L-lactide) (PLLA/PLA), poly(L-lactide-co-glycolide) (PLLA/PGA), poly(D,L-lactide-co-glycolide) (PLA/PGA), poly(glycolide-co-trimethylene carbonate) (PGA/PTMC), polydioxanone (PDS), Polycaprolactone (PCL), polyhydroxybutyrate (PHBT), poly(phosphazene)poly(D,L-lactide-co-caprolactone) PLA/PCL), poly(glycolide-co-caprolactone) (PGA/PCL), poly(phosphate ester), polyethylene glycols (PEG), and the like.

Also, the blood access device 10, the porous polymeric portions 14, 18, 22, may be treated with any known or useful bioactive agent or drug including without limitation the following: anti-thrombogenic agents (such as heparin, heparin derivatives, urokinase, and PPack (dextrophenylalanine proline arginine chloromethylketone); anti-proliferative agents (such as enoxaprin, angiopeptin, or monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-miotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin, prostaglandin inhibitors, platelet inhibitors and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vascoactive mechanisms.

While various embodiments of the present invention are specifically illustrated and/or described herein, it will be appreciated that modifications and variations of the present invention may be effected by those skilled in the art without departing from the spirit and intended scope of the invention. Further, any of the embodiments or aspects of the invention as described in the claims or in the specification may be used with one and another without limitation.

What is claimed is:

1. A blood access device comprising:
a radially distensible support structure having an open lattice structure defining a support wall having interstitial open areas and defining exterior and luminal wall surfaces; and
a first porous polymeric portion having a plurality of pores, the first porous polymeric portion being securably disposed over at least a portion of the exterior wall portion of the support structure.

2. The blood access device of claim 1, further comprising:
a second porous polymeric portion having a plurality of pores, the second porous polymeric portion being securably disposed over at least a portion of the luminal portion of the support structure.

3. The blood access device of claim 1, wherein the first porous polymeric portion and the second porous polymeric portion are a unitary porous polymeric portion.

4. The blood access device of claim 2, wherein the first and/or the second polymeric portions encapsulate the support structure.

5. The blood access device of claim 2, wherein the first polymeric portion and/or the second polymeric portion are disposed within interstitial open areas of the support structure.

6. The blood access device of claim 2, further comprising:
a biodegradable and/or bioabsorbable material disposed within the pores of the first polymeric portion and/or pores of the second polymeric portion.

7. The blood access device of claim 2, further comprising a porous layer or coating of expanded polytetrafluoroethylene disposed over a luminal surface of the second polymeric portion.

8. The blood access device of claim 1, wherein the support structure is a self-expanding support structure.

9. The blood access device of claim 1, wherein the support structure is selected from the group consisting of a filament-based structure, an open slotted structure, a mesh or fenestrated structure, and combinations thereof.

10. The blood access device of claim 9, wherein the filament based structure is selected from the group consisting of a braided structure, a knitted structure, a wound structure, a helical structure, a zig-zag structure, and combinations thereof.

11. The blood access device of claim 1, wherein the support structure comprises nitinol, stainless steel, cobalt-based alloy such as Elgiloy®, platinum, gold, titanium, tantalum, niobium, polymeric materials and combinations thereof.

12. The blood access device of claim 1, wherein the support structure comprises nitinol.

13. The blood access device of claim 1, wherein the support structure comprises vapor deposited nitinol.

14. The blood access device of claim 1, wherein the first polymeric portion comprises an elastomeric material.

15. The blood access device of claim 2, wherein the second polymeric portion comprises an elastomeric material.

16. The blood access device of claim 2, wherein the first and second polymeric portions comprise an elastomeric material.

17. The blood access device of claim 16, wherein the elastomeric material of the first polymeric portion is the same as the elastomeric material of the second polymeric portion.

18. The blood access device of claim 16, wherein the elastomeric material of the first polymeric portion is different from the elastomeric material of the second polymeric portion.

19. The blood access device of claim 14, wherein the elastomeric material is selected from the group consisting of styrene isobutylene styrenes, natural rubbers, silicones, polyurethanes, and combinations, co-polymers, block polymers and random polymers thereof.

20. The blood access device of claim 14, wherein the elastomeric material comprises styrene isobutylene styrene polymer and co-polymers, block polymers and random polymers thereof.

21. The blood access device of claim 2, wherein the first and second polymeric portions comprise textile portions, non-textile portions, and combinations thereof.

22. The blood access device of claim 2, wherein the first and second polymeric portions comprise filament spun portions, wherein the filaments comprise an elastomeric material.

23. The blood access device of claim 22, wherein the elastomeric material comprises styrene isobutylene styrene polymer and co-polymers, block polymers and random polymers thereof.

24. The blood access device of claim 2, further comprising a therapeutic agent disposed within the pores of the first polymeric portion and/or pores of the second polymeric portion.

25. The blood access device of claim 2, wherein the pores of the first polymeric portion and/or the second polymeric portion have a pore size from about 40 microns to about 150 microns.

26. The blood access device of claim 2, wherein the wall of the support structure has a thickness from about 0.0005 inches (0.01 mm) to about 0.008 inches (0.2 mm).

27. The blood access device of claim 2, wherein the wall of the support structure has a thickness from about 0.001 inches (0.03 nm) to about 0.004 inches (0.1 mm).

28. The blood access device of claim 2, wherein the first and second polymeric portions have an individual or combined thickness from about 0.002 inches (50 microns) to about 0.06 inches (1.5 mm).

29. A blood access for dialysis comprising:
a radially distensible support structure having an open lattice structure defining a support wall having interstitial open areas and defining exterior and luminal wall surfaces;
a first porous polymeric portion having a plurality of pores, the first porous polymeric portion being securably disposed over at least a portion of the exterior wall portion of the support structure;
a second porous polymeric portion having a plurality of pores, the second porous polymeric portion being securably disposed over at least a portion of the luminal portion of the support structure; and
a biodegradable and/or bioabsorbable material disposed within the pores of the first polymeric portion and/or pores of the second polymeric portion.

30. The device of claim 29, wherein the support structure comprises nitinol.

31. The device of claim 29, wherein the first and the second polymeric portions comprises elastomeric styrene-isobutylene-styrene.

32. A system for providing blood access for dialysis, comprising:
a radially distensible support structure having an open lattice structure defining a support wall having interstitial open areas and defining exterior and luminal wall surfaces;
a first porous polymeric portion having a plurality of pores, the first porous polymeric portion being securably disposed over at least a portion of the exterior wall portion of the support structure;
a second porous polymeric portion having a plurality of pores, the second porous polymeric portion being securably disposed over at least a portion of the luminal portion of the support structure;
a biodegradable and/or bioabsorbable material disposed within the pores of the first polymeric portion and/or pores of the second polymeric portion; and
a delivery device for transluminally delivering the blood access within a bodily lumen.

33. A method of reducing arteriovenous fistula maturation comprising:
cutting or severing a vein into a first and a second portion, each portion having open ends;
ligating the open end of the first vein portion;
inserting a blood access device through the open end of the second vein portion; and
anastomosing the open end of the second vein to an artery.

34. The method of claim 33, wherein the blood access device comprises:
a radially distensible support structure having an open lattice structure defining a support wall having interstitial open areas and defining exterior and luminal wall surfaces;
a first porous polymeric portion having a plurality of pores, the first porous polymeric portion being securably disposed over at least a portion of the exterior wall portion of the support structure;
a second porous polymeric portion having a plurality of pores, the second porous polymeric portion being securably disposed over at least a portion of the luminal portion of the support structure; and
a biodegradable and/or bioabsorbable material disposed within the pores of the first polymeric portion and/or pores of the second polymeric portion.

35. The method of claim 33, wherein the blood access device is useable prior to complete maturation of the arteriovenous fistula.

36. The method of claim 35, wherein the blood access device is useable within about one week after being implanted.

37. A method of creating an arteriovenous fistula comprising:
cutting or severing a vein into a first and a second portion, each portion having open ends;
ligating the open end of the first vein portion;
inserting a blood access device through the open end of the second vein portion; and
anastomosing the open end of the second vein to an artery.

38. The method of claim 37, wherein the blood access device comprises:
a radially distensible support structure having an open lattice structure defining a support wall having interstitial open areas and defining exterior and luminal wall surfaces;
a first porous polymeric portion having a plurality of pores, the first porous polymeric portion being securably disposed over at least a portion of the exterior wall portion of the support structure;
a second porous polymeric portion having a plurality of pores, the second porous polymeric portion being securably disposed over at least a portion of the luminal portion of the support structure; and
a biodegradable and/or bioabsorbable material disposed within the pores of the first polymeric portion and/or pores of the second polymeric portion.

39. The method of claim 38, wherein the blood access device is useable prior to complete maturation of the arteriovenous fistula.

40. The method of claim 39, wherein the blood access device is useable within about one week after being implanted.

41. The method of claim 37, wherein the support structure tends to limit the expansion or an internal diameter of the vein proximal to the site of implantation of the blood access device.

42. The method of claim 37, wherein the blood access device encompasses a minor portion of the second vein portion.

43. The method of claim 37, wherein the blood access device encompasses a major portion of the second vein portion.

44. A method for making a blood access device for use in dialysis, comprising:
providing a porous polymeric substrate;
disposing a radially distensible support structure over the porous polymeric structure,
providing another porous polymeric substrate over the support structure; and
securing the substrates to one and the other anchor to the support structure.

45. The method of claim 44, where the steps of providing the porous polymeric substrates further comprise providing an elastomeric material.

46. The method of claim 45, further comprising the step of spinning or spraying filaments of the elastomeric material.

47. The method of claim 46, wherein the elastomeric material styrene isobutylene styrene polymer and co-polymers, block polymers and random polymers thereof.
